(19) United States
(12) Patent Application Publication
(10) Pub. No.: US 2005/0118351 A1
(43) Pub. Date: Jun. 2, 2005

(54) Method of forming film, electro-optic device and electronic equipment

(75) Inventor: Hirotsuna Miura, Suwa-gun (JP)

Correspondence Address: OLIFF & BERRIDGE, PLC, P.O. BOX 19928, ALEXANDRIA, VA, 22320, US

(73) Assignee: Seiko Epson Corporation, Tokyo (JP)

(21) Appl. No.: 10/959,183

(22) Filed: Oct. 7, 2004

(30) Foreign Application Priority Data
Oct. 28, 2003 (JP) 2003-367505

(51) Int. Cl. A61F 13/15; A61F 13/20; H05H 1/32

(52) U.S. Cl. 427/540

(57) ABSTRACT

Exemplary embodiments provide a method of forming a film which is capable of forming uniform film with no or substantially no irregularities. A method of forming an orientation film of liquid crystal molecules includes applying a liquid material from an ink-jet head to a substrate in which a driving electrode of a liquid crystal layer is formed. The liquid material is heated below a boiling point to facilitate fluidization by supplying a current to the driving electrode before the application or during the application of the liquid material. Moreover, the liquid material is heated to no less than the boiling point by supplying the current to the driving electrode after the application of the liquid material, and is dried. In addition, it is also possible to control the dry condition by supplying the different currents to a plurality of driving electrodes.

### BACKGROUND OF THE INVENTION

1. Field of Invention

Exemplary embodiments of the present invention relate to a method of forming a film, an electro-optic device, and electronic equipment.

2. Description of Related Art

A related art liquid crystal display device can be used as a light modulation device in a projector, a direct vision type display device in a cellular phone, or the like. This related art liquid crystal display device includes a liquid crystal layer interposed between a pair of substrates that are arranged facing each other. Inside the pair of the substrates, a transparent electrode to apply an electric field to the liquid crystal layer is formed. Inside the electrode, an orientation film, which controls the arrangement of liquid crystal molecules when no electric field is applied, is formed. Then, image display is carried out based on changes of the arrangement of the liquid crystal molecules when no electric field is applied and when electric field is applied.

The above described orientation film is formed of polymer materials, such as polyimide. In order to form the orientation film, the liquid material containing an orientation film formation material is applied on the substrate, and the applied liquid material is heat-treated to obtain a dry film. Then, the orientation film can be formed by carrying out rubbing processing to the surface of the dry film. In addition, as the method of applying a liquid material on a substrate, a spin coating method, a dipping method, a spraying method, a printing method, a droplet discharging method, or the like can be used, for example.

Among these, the droplet discharging method is the method of applying a liquid material by discharging a plurality of droplets on the substrate. In this case, the discharged droplet spreads wet on the substrate, and joints with adjoining droplets, and it is thereby in a condition that the liquid material is being applied. This droplet discharging method has an advantage in that a predetermined amount of liquid material can be applied to a predetermined position accurately, and the liquid material can be used efficiently.

A related art method is disclosed in Japanese Unexamined Patent Publication No. H9-105938.

### SUMMARY OF THE INVENTION

However, at the time of the application of the liquid material by the droplet discharging method, there are cases in which a part of a solvent of the droplet evaporates before the discharged droplet spreads wet. Accordingly, the viscosity of the droplet increases and the fluidity decreases. In this case, there is a problem in that it is difficult to form the orientation film uniformly.

Moreover, in case that the orientation film is formed on a large substrate, the liquid material is applied over a plurality of lines by making a head of the droplet discharging device to make a new line. In this case, if the droplet fluidity decreases, there is a problem in that a mixing defect of the liquid material occurs in the boundary portion of adjacent lines, and a line feed streak appears in the portion. This line feed streak decreases the display quality of the liquid crystal display device.

On the other hand, when the applied liquid material is being dried, the steam partial pressure of a solvent becomes high in the center portion on the substrate, and the steam partial pressure becomes low in the periphery portion. For this reason, drying is delayed in the center portion, while it dries up promptly in the periphery portion, and there is a problem in that dryness irregularity occurs in the orientation film. This dryness irregularity also decreases display quality of the liquid crystal display device.

In addition, Japanese Unexamined Patent Publication No. H9-105938 discloses a method of forming a uniform orientation film by controlling time after applying an orientation film formation solution and before the heating is started. However, because infrared rays or microwaves that have an amount of heat that is non-uniform are used for the heating, it is difficult to form the orientation film uniformly.

Exemplary embodiments of the present invention address or solve the above and/or other problems, and provide a method of forming a film enabling the formation of a film which is uniform and has no or substantially no irregularity.

Moreover, exemplary embodiments provide a liquid crystal display device and electronic equipment that are excellent in display quality.

In order to address or attain the above, a method of forming a film according to exemplary embodiments of the present invention includes applying a liquid material and forming a film on a substrate in which an electric conduction layer is formed. A current is supplied to the electric conduction layer before an application or during an application of the liquid material.

According to this structure, the applied liquid material can be heated by having the electric conduction layer generate heat. Then, because the electric conduction layer is preheated before the application or during the application of the liquid material, the increase of viscosity due to the decreased temperature of the applied liquid material is reduced or suppressed. This facilitates fluidization of the liquid material, and the liquid material spreads wet in a uniform thickness. Moreover, even when applying the liquid material over a plurality of lines, the occurrence of a line feed streak can be reduced or prevented because the liquid material is mixed favorably in the boundary portion of the adjacent lines. Accordingly, a uniform film can be formed.

Moreover, it is desirable that the current supply to the electric conduction layer is carried out so that the temperature of the electric conduction layer may be less than the boiling point of the liquid material.

According to this structure, the increase of viscosity due to the evaporation of the liquid material is reduced or suppressed. This facilitates fluidization of the liquid material, and thereby a uniform film can be formed.

On the other hand, another exemplary method of forming a film on a substrate in which an electric conduction layer is formed includes applying a liquid material. Current is supplied to the electric conduction layer after the application of the liquid material.

According to this structure, the liquid material can be heated uniformly compared with the case where infrared rays, microwaves, or the like are used, and thus a film without irregularity can be formed. Moreover, heating device that irradiates infrared rays, microwaves, or the like is also unnecessary, and thus the equipment cost can be reduced. Furthermore, because the liquid material is heated by the electric conduction layer adjacent to the applied liquid material, it is possible to dry the liquid material promptly with a small amount of heat, and thus reduction of energy consumption and reduction of the drying time can be realized.

Moreover, it is desirable that the current supply to the electric conduction layer is carried out, so that the temperature of the electric conduction layer may become no less than the boiling point of the liquid material.

According to this structure, a film without the dryness irregularity can be formed.

Moreover, it is desirable that the electric conduction layer is provided with a plurality of electrically isolated conduction portions, and more current is supplied to the electric conduction portion arranged in the center portion of the substrate, than to the electric conduction portion arranged in the periphery portion on the substrate.

According to this structure, the drying speed of the liquid material on the substrate can be made uniform because the liquid material applied to the center portion on the substrate is heated strongly. Accordingly, an orientation film without irregularity can be formed.

Moreover, the electric conduction layer may be provided with a plurality of electrically isolated conduction portions, and more current is supplied to the electric conduction portion arranged in a region during the application or after the application of the liquid material, than to the electric conduction portion arranged in a region before the application of the liquid material.

According to this structure, the drying processing can be carried out immediately to the region during the application or after the application of the liquid material, and the drying time can be shortened. Moreover, recoating of the liquid material can be also carried out efficiently.

Moreover, it is desirable that the electric conduction layer is an electrode layer that drives an image display element.

According to this structure, the liquid material can be heated uniformly because the electrode is formed in almost an entire film formation region. Accordingly, a uniform film can be formed.

Moreover, the electric conduction portion may be a scanning electrode or a signal electrode in a passive matrix type electro-optic device.

According to this structure, the current can be easily supplied from both end portions of each electrode formed in a striped shape.

Moreover, the electric conduction layer may be a light-shielding film formed around an image display element. Moreover, the electric conduction portion may be a plurality of light-shielding portions that electrically isolate the light-shielding film formed in the surrounding of the image display element.

The above can also be addressed or attained with these structures.

On the other hand, the electro-optic device according to exemplary embodiments of the present invention is manufactured using the above described methods of forming a film.

According to this structure, an electro-optic device that is excellent in display quality can be provided because a film, which is uniform and has no irregularity, can be formed.

On the other hand, electronic equipment according to exemplary embodiments of the present invention includes the above described electro-optic device.

According to this structure, electronic equipment that is excellent in display quality can be provided.

### BRIEF DESCRIPTION OF THE DRAWINGS

FIG. 1 is a perspective view of a liquid crystal display device;

FIG. 2 is a front sectional view taken along plane A-A of FIG. 1;

FIG. 3 is a perspective view of a droplet discharging device;

FIG. 4 is a side sectional view of an ink-jet head;

FIG. 5 is a schematic of a method of applying a liquid material;

FIG. 6 is a schematic of a black matrix;

FIG. 7 is a schematic of an exemplary modification of the black matrix; and

FIG. 8 is a perspective view of a cellular phone.

### DETAILED DESCRIPTION OF PREFERRED EMBODIMENTS

Exemplary embodiments of the present invention are described below with reference to accompanying drawings. In addition, in each drawing used for the following description, the scale of each member is changed suitably in order to make each member a recognizable size.

In addition, in the present specification, the liquid crystal layer side in the component member of the liquid crystal display device will be referred to as an inner side.

### First Exemplary Embodiment

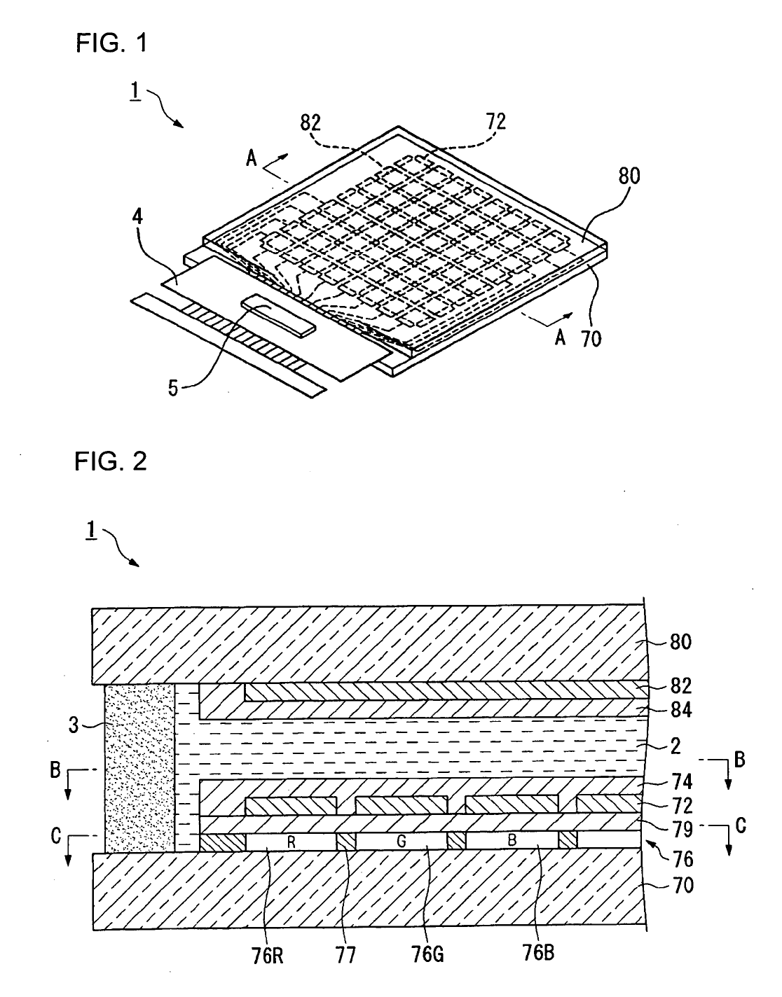

A first exemplary embodiment of the present invention is described with reference to FIG. 1 through FIG. 5. The method of forming a film of the first exemplary embodiment is the method of forming an orientation film 74 in a liquid crystal display device 1 shown in FIG. 2. The liquid material containing an orientation film 74 formation material is applied on a substrate 70, and an orientation film 74 is formed by drying the applied liquid material, and the liquid material is heated by supplying the current to a driving electrode 72 of a liquid crystal layer 2 before the application, during the application and after the application of the liquid material.

### Exemplary Liquid Crystal Display Device

FIG. 1 is a perspective view of a liquid crystal display device, and FIG. 2 is a front sectional view taken along plane A-A of FIG. 1. The liquid crystal display device 1 shown in FIG. 2 is constituted interposing the liquid crystal layer 2 with a lower substrate 70 and an upper substrate 80. In addition, although a passive matrix type liquid crystal display device is described as an example in the present exemplary embodiment, the present invention can be also applied to an active matrix type liquid crystal display device, for example.

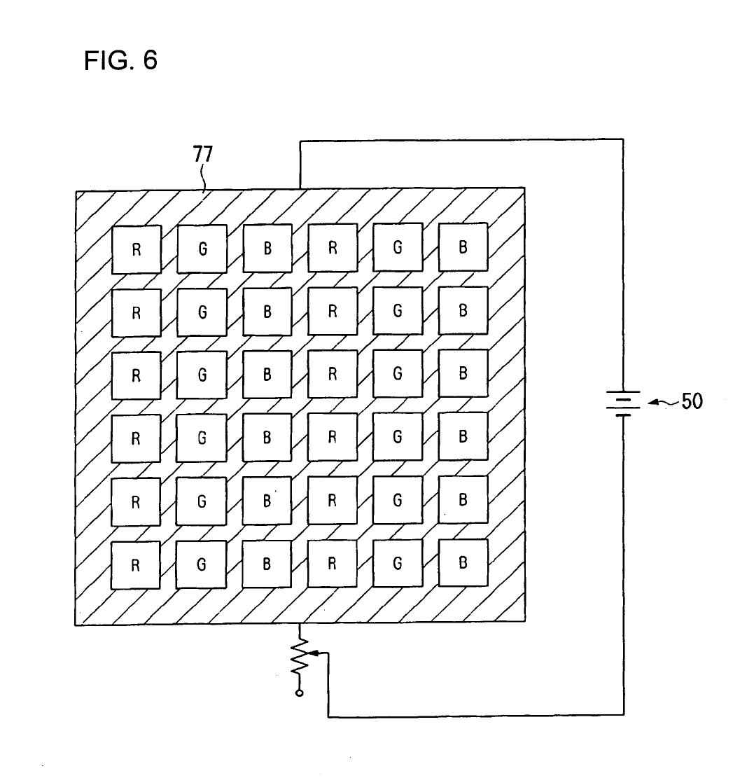

As shown in FIG. 2, in the liquid crystal display device 1, the lower substrate 70 and the upper substrate 80 made of transparent material, such as glass, are arranged facing to each other. A color filter layer 76 is formed in the inner side of the lower substrate 70. In this color filter layer, a plurality of color filters R, G, and B which transmit each color light of red, green, or blue are arranged in a matrix form (refer to FIG. 6). Moreover, in order to reduce or prevent color mixing of the color light which passes through each color filter, a black matrix (light-shielding film) 77 made of a black material, such as chromium metal, is arranged around each of color filters R, G, and B shown in FIG. 2. Furthermore, in the inner side of the color filter layer 76, a protection film 79 for the color filter layer is formed. In addition, the color filter layer 76 and the protection film 79 thereof may be formed in the inner side of the upper substrate 80.

In the inner side of the lower substrate 70 and the upper substrate 80, driving electrodes 72 and 82 to apply an electric field to the liquid crystal layer are formed. These driving electrodes 72 and 82 are formed of the transparent conductive material, such as ITO, in a striped shape. Then, as shown in FIG. 1, the driving electrode 72 of the lower substrate 70 and the driving electrode 82 of the upper substrate 80 are arranged as to intersect perpendicularly. In addition, each of the driving electrodes 72 and 82 is coupled to a driver IC5, and the scanning signal is provided from this driving IC5 to one driving electrode, and at the same time a data signal is provided to other driving electrode. Moreover, each of the color filters R, G, and B shown in FIG. 2 is arranged near the intersection of the both electrodes to form a dot region, and one pixel (image display element) region is constituted by three dot regions having a color filter which transmits a different color light.

Furthermore, as shown in FIG. 2, orientation films 74 and 84 are formed as to cover each of the driving electrodes 72 and 82. These orientation films 74 and 84 control the orientation condition of the liquid crystal molecules when no electric field is applied. The orientation films 74 and 84 are formed of organic polymer materials, such as polyimide, and rubbing processing is carried out to the surface thereof. Accordingly, when no electric field is applied, the liquid crystal molecules near the surface of the orientation films 74 and 84 are oriented as to be approximately in parallel with the orientation films 74 and 84, with the longitudinal direction thereof being aligned to the rubbing processing direction. In addition, the rubbing processing is carried out to each of the orientation films 74 and 84, so that the orientation direction of the liquid crystal molecule near the surface of the orientation film 74 and the orientation direction of the liquid crystal molecule near the surface of the orientation film 84 may deviate by a predetermined angle.

Accordingly, the liquid crystal molecules are deposited spirally along the thickness direction of the liquid crystal layer 2.

The space between the lower substrate 70 and the upper substrate 80 is provided by the diameter of a bead-shaped spacer (not shown) arranged between the both substrates, and, for example, is maintained at approximately 5 μm. Moreover, in the both substrates 70 and 80, the periphery portions are bonded by a sealing material 3 made of adhesives, such as a thermosetting type and an ultraviolet-cured type. Then, the liquid crystal layer 2 is sealed in the space surrounded by the both substrates 70 and 80 and the sealing material 3. Nematic liquid crystal or the like is adopted for this liquid crystal layer 2, and a super twisted nematic (STN) mode is adopted as the operation mode of the liquid crystal display device 1. In addition, it is also possible to adopt liquid crystal material other than the above described ones, and operation modes other than the above described one can be also adopted.

In addition, in the outside of the lower substrate 70 and the upper substrate 80, polarizing plates (not shown) are arranged with the mutual polarization axes (transmission axis) been deviated by a predetermined angle. Moreover, a backlight (not shown) is arranged in the outside of an incidence side polarizing plate.

Then, the light irradiated from the backlight is converted into a linearly polarized light along the polarization axis of the incidence side polarizing plate, and enters the liquid crystal layer 2 from the lower substrate 70. This linearly polarized light, in the process of passing through the liquid crystal layer 2 in the condition of no electric field being applied, rotates by a predetermined angle along the twist direction of the liquid crystal molecules, and passes through the outgoing side polarizing plate. Accordingly, white display is carried out when no electric-field is applied (normally white mode). On the other hand, when an electric field is applied to the liquid crystal layer 2, the liquid crystal molecules will re-orientate perpendicularly to the orientation films 74 and 84 along the electric field direction. In this case, the linearly polarized light which entered the liquid crystal layer 2 does not rotate, therefore, will not pass through the outgoing side polarizing plate. Accordingly, black display is carried out when no electric-field is being applied. In addition, it is also possible to carry out gray-scale display according to the strength of the applied electric field. Moreover, because a white light irradiated from the backlight is converted into a colored light in the process of passing through the color filter layer 76, it is also possible to carry out color image display by an additive mixture of color stimuli.

### Exemplary Droplet Discharging Device

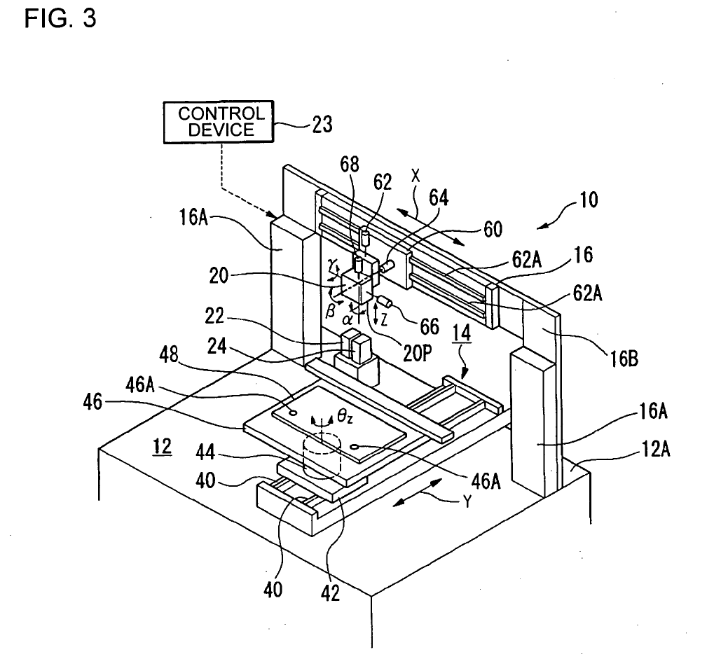

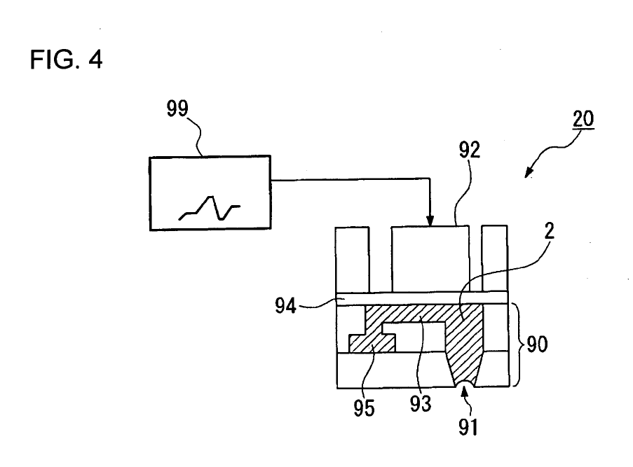

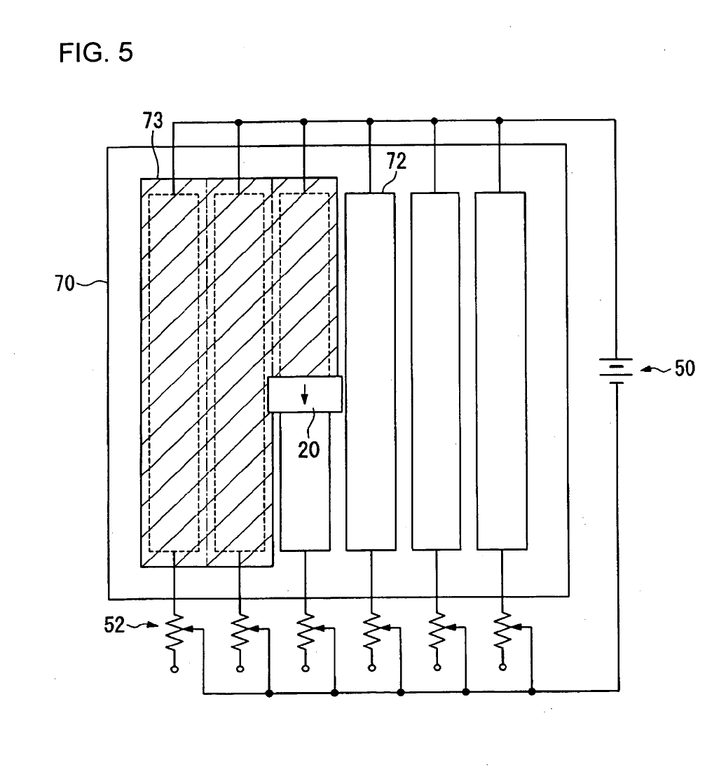

The present exemplary embodiment relates to a method of forming the above described orientation films 74 and 84. The orientation films 74 and 84 are formed by discharging the component material solution thereof from the droplet discharging device. The droplet discharging device is described using FIG. 3 and FIG. 4.

FIG. 3 is a perspective view of the droplet discharging device. In FIG. 3, an X direction is the right-and-left direction of a base 12, a Y direction is the back and forth direction, and a Z direction is the up and down direction. The droplet discharging device 10 is constituted mainly by an ink-jet head (hereinafter “head”) 20 and a table 46 which installs a substrate 48. In addition, provision is made to control the operation of the droplet discharging device 10 by a control device 23.

The table 46 which installs the substrate 48 is allowed to move and position in the Y direction by a first moving device 14, and is allowed to oscillate and position in the θz direction by a motor 44. On the other hand, the head 20 is allowed to move and position in the X direction by a second moving device, and is allowed to move and position in the Z direction by a linear motor 62. Moreover, the head 20 is allowed to oscillate and position in α, β, and γ directions by motors 64, 66, and 68, respectively. Accordingly, the droplet discharging device 10 is designed to be able to control accurately the relative position and attitude of an ink discharging face 20P of the head 20 and the substrate 48 on the table 46.

Here, an example of the structure of the head 20 is described with reference to FIG. 4. FIG. 4 is a side sectional view of the ink-jet head. The head 20 discharges ink 2 from a nozzle 91 with a droplet discharging method. As the droplet discharging method, various kinds of related art or well-known technologies, such as a piezo method of discharging ink by using a piezo actuator as a piezoelectric actuator, and a method of discharging ink with bubbles generated by heating the ink, can be used. Among these, the piezo method has an advantage of not giving influence to the material composition or the like because no heat is applied to the ink.

Then, as the head 20 of FIG. 4, the above described piezo method has been adopted.

In a head main part 90 of the head 20, a reservoir 95 and a plurality of ink chambers 93 branched from the reservoir 95 are formed. The reservoir 95 is a flow channel for providing ink to each of the ink chambers 93. Moreover, the lower end face of the head main part 90 is provided with a nozzle plate which constitutes an ink discharging face. In the nozzle plate, a plurality of nozzles 91 which discharge ink are opened corresponding to each of the ink chambers 93. Then, the ink channel is formed toward the corresponding nozzle 91 from each of the ink chambers 93. On the other hand, the upper end face of the head main part 90 is provided with an oscillation plate 94. In addition, the oscillation plate 94 constitutes the wall surface of each of the ink chambers 93. In the outside of the oscillation plate 94, a piezo actuator 92 is provided corresponding to each of the ink chambers 93. The piezo actuator 92 is the one that interposes a piezoelectric material, such as quartz, with a pair of electrodes (not shown). The pair of electrodes is coupled to a driving circuit 99.

Then, when a voltage is applied to the piezo actuator 92 from the driving circuit 99, the piezo actuator 92 will expansion-deform or contraction-deform. When the piezo actuator 92 contraction-deforms, the pressure in the ink chamber 93 will decrease and the ink 2 will flow into the ink chamber 93 from the reservoir 95. Moreover, if the piezo actuator 92 expansion-deforms, the pressure in the ink chamber 93 increases, and the ink 2 will be discharged from the nozzle 91. In addition, the deformation amount of the piezo actuator 92 can be controlled by changing the applied voltage. Moreover, the deformation speed of the piezo actuator 92 can be controlled by changing the frequency of the applied voltage. That is, the discharging conditions of the ink 2 can be controlled by controlling the applied voltage to the piezo actuator 92.

In addition, a capping unit 22 shown in FIG. 3 is the one that caps the ink discharging face 20P at the time of standby of the droplet discharging device 10, in order to reduce or prevent the ink discharging face 20P in the head 20 from being dried. Moreover, a cleaning unit 24 is the one that sucks the inside of the nozzle in order to remove the nozzle clogging in the head 20. In addition, the cleaning unit 24 can also carry out the wiping of the ink discharging face 20P in order to remove the dirt of the ink discharging face 20P in the head 20.

### Exemplary Application Method

A method of applying a liquid material containing an orientation film formation material using the above described droplet discharging device is described using FIG. 5. FIG. 5 is a schematic of the method of applying a liquid material, and is a planar sectional view taken along plane B-B of FIG. 2. In addition, hereinafter the case where the orientation film is formed inside the lower substrate 70 is described as an example. However, it is also possible to form the orientation film inside the upper substrate with the same method.

In the present exemplary embodiment, the current is supplied to the driving electrode 72 formed in the lower substrate 70, and the Joule heat is generated by the electrical resistance thereof to heat the liquid material. Then, as shown in FIG. 5, each driving electrode 72 is coupled to a power supply 50. Specifically, a plurality of driving electrodes 72 formed in a striped shape are coupled in series to a variable resister 52, respectively, and these will be further coupled in parallel with respect to the power supply 50. As for this power supply 50, it is desirable to adopt the one which can change the applying voltage without constraint.

Moreover, as for the variable resister 52, it is preferable to adopt one having a resistance that can be changed from zero to infinite. These can adjust the amount of the current supplied to each driving electrode 72 without constraint.

Then, the current is supplied to all the driving electrodes 72 to preheat each driving electrode 72. In this case, the amount of the supply current to each driving electrode 72 is adjusted so that the temperature of each driving electrode 72 may become the temperature below the boiling point of the solvent of the liquid material 73 to be applied.

On the other hand, soluble polyimide which is the orientation film formation material is dissolved in a solvent, such as gamma-butyl-lactone (boiling point of 204° C.) or the like, and the liquid material 73 to be applied is made. Then, this liquid material 73 is discharged on the surface of the driving electrode 72 from the ink-jet head 20 of the droplet discharging device. In addition, in the width direction of the head 20 described above, a plurality of nozzles are arranged in one row or in the staggered form.

Then, the liquid material 73 can be applied in a planar form by discharging the liquid material from each nozzle of the head 20, while the head 20 is being moved in the direction orthogonal to the width direction. In addition, in case that the width of the orientation film formation region in the lower substrate 70 is equal to the width of the head 20, the liquid material can be applied to the whole orientation film formation region by sweeping the head 20 only once.

Because each driving electrode 72 is preheated, the increase of viscosity due to the temperature decrease of the liquid material 73 is reduced or suppressed. In addition, because the preheating is carried out at the temperature (for example, 50° C.) below the boiling point of the liquid material 73 solvent, the increase of viscosity due to the evaporation of the solvent is also reduced or suppressed. This facilitates fluidization of the discharged liquid material 73, and the liquid material 73 spreads wet in a uniform thickness. Accordingly, a uniform orientation film can be formed. It is desirable to apply the liquid material 73 under the condition that the steam partial pressure of the solvent in the substrate periphery is made high. In this case, because natural evaporation of the solvent can also be reduced or suppressed, a more uniform orientation film can be formed.

On the other hand, as shown in FIG. 5, in case that the width of the orientation film formation region is larger than the width of the head 20, the liquid material 73 is applied to the whole orientation film formation region by dividing the orientation film formation region into a plurality of lines and sweeping the head 20 per each line. In this case, it is desirable to apply the liquid material 73 by sweeping the head 20 in the longitudinal direction of the driving electrode 72 that is formed in a striped shape. In addition, actually, the width of the driving electrode 72 is remarkably smaller than the width of the head 20, therefore, the liquid material 73 will be applied to the surfaces of a plurality of driving electrodes 72 by one time sweep.

Also in this case, because each driving electrode 72 is preheated, the discharged liquid material spreads wet favorably. Then, the liquid material applied to the adjacent line is mixed favorably in the mutual boundary portion. Accordingly, the occurrence of so-called line feed streaks can be reduced or prevented. Accordingly, a liquid crystal display device that is excellent in display quality can be provided.

As described above, the amount of the current supplied to each driving electrode 72 can be adjusted without constraint. Then, as for the driving electrode 72 that is arranged in a line during the application or after the application of the liquid material 73, the amount of the supply current may be increased. In this case, the amount of the supply current is increased so that the temperature of the driving electrode 72 may become the temperature no less than the boiling point of the liquid material 73. Accordingly drying processing can be carried out to the line promptly during the application or after the application of the liquid material 73, and the drying time can be shortened. Moreover, it is also possible to have completed the drying processing to the first application line at the time when the liquid material has been applied to the whole orientation film formation region. In this case, a recoating of the liquid material can be carried out promptly from the first application line, and thus the recoating can be carried out efficiently.

### Exemplary Drying Method

A method of drying the liquid material applied to the whole orientation film formation region is described below.

At the time when the application of the liquid material 73 is complete to the whole orientation film formation region, the amount of the supply current to each driving electrode 72 is increased so that the temperature of all the driving electrodes 72 may become the temperature no less than the boiling point of the liquid material (for example, 220° C.). Accordingly, the liquid material 73 is heated, the solvent evaporates, and a dry film is formed.

In addition, because the driving electrode 72 is formed in almost the whole of the orientation film formation region, the applied liquid material 73 can be heated equally. Accordingly, an orientation film without irregularity can be formed as compared with the case of heating with an oven, a hot plate, an infrared lamp, or the like. In addition, the heating device, such as an oven, a hot plate, and an infrared lamp are also unnecessary, and thus the equipment cost can be reduced. On the other hand, because the liquid material 73 is heated by the driving electrode 72 arranged directly under the orientation film, the liquid material 73 can be dried promptly with the small amount of heat, thereby enabling the reduction of energy consumption and the reduction of drying time. In this case, because the liquid material 73 can be heated without making the lower substrate 70 being in a high temperature, disconnection or the like due to expansion deformation of the lower substrate 70 can be reduced or prevented.

When the solvent evaporates from one part of the liquid material 73, the steam partial pressure of the solvent will increase and evaporation of the solvent in the peripheral portion will be reduced or suppressed. For this reason, the drying speed of the liquid material 73 in the center portion of the orientation film formation region tends to be slow as compared with the peripheral portion. Then, it is desirable that the amount of the current supplied to the driving electrode 72, which is arranged in the center portion of the orientation film formation region, is made more than the amount of the current supplied to the driving electrode 72 arranged in the peripheral portion. Accordingly the liquid material 73 applied to the center portion of the orientation film formation region is heated strongly to facilitate the drying, so that the drying speed in the orientation film formation region can be made uniform. Therefore, an orientation film without irregularity can be formed.

### Second Exemplary Embodiment

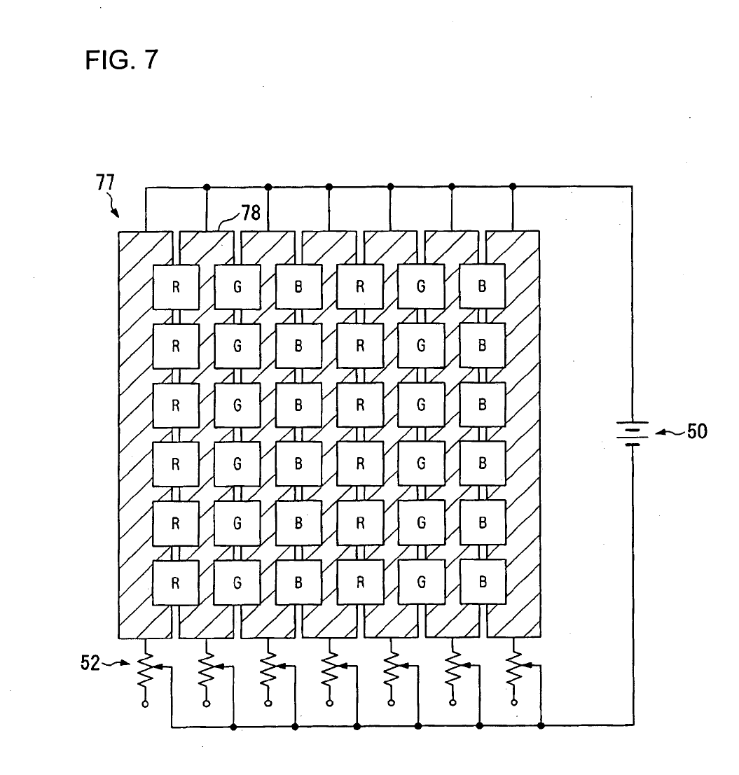

A second exemplary embodiment according to the present invention is described using FIG. 6 and FIG. 7. FIG. 6 is a schematic of a black matrix, and is a planar sectional view taken along plane C-C of FIG. 2. The method of forming a film of the second exemplary embodiment differs from the first exemplary embodiment in that the liquid material is heated by supplying the current to the black matrix (light-shielding film) 77. In addition, a detailed description regarding portions that become the same structure as the first exemplary embodiment will be omitted.

### Exemplary Application Method

In this exemplary embodiment, the current is supplied to the black matrix 77 formed in the lower substrate, and Joule heat is generated by the electrical resistance to heat the liquid material. In addition, the general black matrix 77 is formed electrically in series. In this case, as shown in FIG. 6, both end portions of the black matrix 77 are coupled to the power supply 50.

FIG. 7 is a schematic of an exemplary modification of the black matrix, and is a planar sectional view taken along the portion corresponding to plane C-C of FIG. 2. The black matrix 77 shown in FIG. 7 is constituted by a plurality of light-shielding portions 78 that are electrically isolated. Each light-shielding portion 78 is formed electrically in series along one side (up and down direction of the page) of the orientation film formation region, and is electrically isolated and formed along other side (right-and-left direction of the page). In this case, like the first exemplary embodiment, each light-shielding portion 78 is coupled in series to the variable resister 52, respectively, and these are coupled in parallel to the power supply 50.

Next, the current is supplied to the black matrix 77. Accordingly, the heat generated in the black matrix 77 shown in FIG. 2 is transferred to each driving electrode 72 through the protection film 79 in order to preheat each driving electrode 72. In addition, the amount of the supply current to the black matrix 77 is adjusted so that the temperature of each driving electrode 72 may be the temperature below the boiling point of the solvent of the liquid material to be applied.

Then, the liquid material containing the orientation film 74 formation material is discharged from the ink-jet head of the droplet discharging device to the surface of the driving electrode 72. At this time, because each driving electrode 72 is preheated, the increase of viscosity of the discharged liquid material is reduced or suppressed, and the liquid material will spread wet with a uniform thickness. Therefore, a uniform orientation film can be formed.

Moreover, in case that the width of the orientation film formation region is larger than the width of the head, the liquid material is applied to the whole orientation film formation region by dividing the orientation film formation region into a plurality of lines like the first exemplary embodiment, and sweeping the head per each line. In addition, in case that the black matrix 77 is formed like FIG. 7, it is desirable to apply the liquid material by sweeping the head in the direction where the light-shielding portion 78 is formed electrically in series. Accordingly, the amount of the supply current can be increased only in the light-shielding portion 78 that is arranged in a line during the application or after the application of the liquid material. In addition, it is desirable to increase the amount of the supply current so that the temperature of the driving electrode to be heated by the light-shielding portion 78 may become the temperature no less than the boiling point of the liquid material. Accordingly, the drying processing can be carried out to a line promptly during the application or after the application of the liquid material, and thus the drying time can be shortened.

Moreover, the recoating of the liquid material can be carried out efficiently.

### Exemplary Drying Method

Next, the liquid material applied to the whole orientation film formation region is dried. Specifically, the amount of the supply current to the black matrix 77 is increased so that the temperature of all driving electrodes may become the temperature no less than the boiling point of the liquid material. In addition, in case that the black matrix 77 is formed like FIG. 7, it is desirable that the amount of the current supplied to the light-shielding portion 78 arranged in the center portion of the orientation film formation region is more than the amount of the current supplied to the light-shielding portion 78 arranged in the peripheral portion. Accordingly, the drying speed in the orientation film formation region can be made uniform, and an orientation film without irregularity can be formed.

As described above, the second exemplary embodiment is configured to supply the current to the black matrix 77 and heat the liquid material before the application and during the application of the liquid material. Accordingly, a uniform orientation film like the first exemplary embodiment can be formed, and moreover the occurrence of line feed streaks can be reduced or prevented. The second exemplary embodiment is configured to supply the current to the black matrix 77 and dry the applied liquid material even after the application of the liquid material. Accordingly, an orientation film without irregularity like the first exemplary embodiment can be formed.

### Exemplary Electronic Equipment

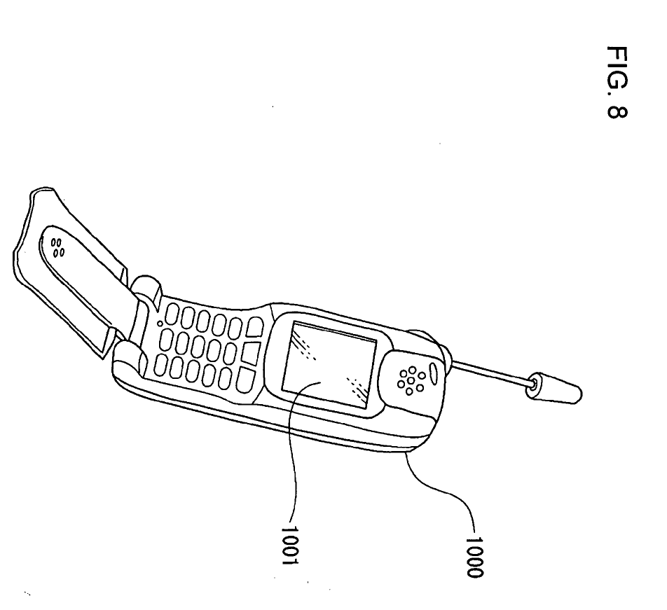

Exemplary electronic equipment manufactured using the method of forming a film according to the present exemplary embodiments is described using FIG. 8. FIG. 8 is a perspective view of a cellular phone. In FIG. 8, a reference numeral 1000 refers to a cellular phone and a reference numeral 1001 refers to a display portion. In this cellular phone 1000, the liquid crystal display device manufactured using the method of forming a film according to the present exemplary embodiment is adopted in the display portion 1001. Therefore, a cellular phone 1000 that is excellent in display quality can be provided at low cost.

In addition, the technical scope of the present invention is not limited to each of the above described exemplary embodiments, and includes various changes added to each of the above described exemplary embodiments within the scope not departing from the purpose thereof.

Namely, specific material, structure, or the like mentioned in each exemplary embodiment is just one example, and can be changed suitably. For example, in the above, a case where the orientation film of a liquid crystal display device is formed has been described, as an example, however, the present invention can be applied to a case where the protection film of a liquid crystal display device is formed, a case where a liquid crystal layer is applied, or the like. Moreover, the present invention can be also applied to a case where a functional film in electro-optic device other than the liquid crystal display device is formed. For example, the present invention can also be applied to a case where the luminescence layer and the hole injection layer of an organic electroluminescence device are formed, or a case where the fluorescent film of a plasma display device is formed.

1. A method of forming a film, comprising:
applying a liquid material on a substrate where an electric conduction layer is formed; and
supplying a current to the electric conduction layer before or during an application of the liquid material.

2. The method of forming a film according to claim 1, the supplying including supplying the current to the electric conduction layer so that the temperature of the electric conduction layer is less than a boiling point of the liquid material.

3. A method of forming a film, comprising:
applying a liquid material on a substrate where an electric conduction layer is formed; and
supplying a current to the electric conduction layer after the application of the liquid material.

4. The method of forming a film according to claim 3, the supplying including supplying the current to the electric conduction layer so that the temperature of the electric conduction layer is no less than a boiling point of the liquid material.

5. The method of forming a film according to claim 3, further including: providing the electric conduction layer with a plurality of electrically isolated conduction portions, and supplying more current to the electric conduction portion arranged in the center portion of the substrate, than to the electric conduction portion arranged in a periphery portion on the substrate.

6. The method of forming a film according to claim 1, further including:
providing the electric conduction layer with a plurality of electrically isolated conduction portions, and supplying more current to the electric conduction portion arranged in a region during the application or after the application of the liquid material, than to the electric conduction portion arranged in a region before the application of the liquid material.

7. The method of forming a film according to claim 1, the electric conduction layer being an electrode layer that drives an image display element.

8. The method of forming a film according to claim 5, the electric conduction portion being at least one of a scanning electrode and a signal electrode in a passive matrix type electro-optic device.

9. The method of forming a film according to claim 1, the electric conduction layer being a light-shielding film formed around an image display element.

10. The method of forming a film according to claim 5, the electric conduction portions being a plurality of light-shielding portions that electrically isolate a light-shielding film formed around an image display element.

11. An electro-optic device manufactured using the method of forming a film according to claim 1.

12. Electronic equipment, comprising:
the electro-optic device according to claim 11.
